(19) United States
(12) Patent Application Publication
(10) Pub. No.: US 2015/0093530 A1
(43) Pub. Date: Apr. 2, 2015

(54) Silicone Article, a Tube and Method of Forming an Article

(71) Applicant: Saint-Gobain Performance Plastics Corporation, Aurora, OH (US)

(72) Inventors: Brian J. Ward, Taunton, MA (US); Ran Ding, Bolton, MA (US); Aijun Zhu, Acton, MA (US)

(21) Appl. No.: 14/498,032

(22) Filed: Sep. 26, 2014

Related U.S. Application Data
(60) Provisional application No. 61/883,404, filed on Sep. 27, 2013.

(51) Int. Cl. C08L 83/04; A61L 31/12

(52) U.S. Cl. CPC C08L 83/04; A61L 31/128
USPC 428/36.9; 524/588

(57) ABSTRACT

The disclosure is directed to a silicone article. The silicone article includes a silicone composition, the silicone composition including a silicone matrix component, a fumed silica filler; and a vinyl-terminated silicone polymer having a viscosity of about 500 centipoise to about 5000 centipoise, wherein the silicone article has a turbidity of less than about 0.3 nephelometric turbidity units (NTU). The disclosure is further directed to a tube and to a method of forming the article.

### FIELD OF THE DISCLOSURE

This disclosure, in general, relates to a silicone article, a tube, and a method of forming an article.

### BACKGROUND

Curable silicone compositions are used in a variety of applications that range from the automotive industry to medical devices. Typical commercial formulations of silicone compositions include a multi-component mixture of a polydiorganosiloxane, a catalyst, and a filler. Often, the commercial formulation is a two-part formulation that is mixed together prior to use. Once the commercial formulation is mixed, the silicone composition is subsequently molded or extruded and vulcanized.

In many cases, articles formed from silicone compositions are needed for various applications. However, typical commercial formulations may include low molecular weight components. Unfortunately, leaching of the low molecular weight components, i.e. byproducts, from the silicone composition may occur when the silicone composition is subjected to heat and a solvent. Turbidity is a measurement of the cloudiness or haziness of a fluid caused by leached byproducts in the solvent. Accordingly, the greater amount of byproducts, the higher the turbidity value. Commercially available formulations typically have a turbidity of greater than about 0.4 nephelometric turbidity units (NTU), as measured by European Pharmacopoeia (EP) 3.1.9. For some applications, it would be advantageous to provide silicone articles with a turbidity value of less than about 0.3 NTU or less.

As such, an improved silicone article and method of forming silicone articles would be desirable.

### SUMMARY

In a particular embodiment, a silicone article is provided. The silicone article includes a silicone composition, the silicone composition including a silicone matrix component, a fumed silica filler; and a vinyl-terminated silicone polymer having a viscosity of about 500 centipoise to about 5000 centipoise, wherein the silicone article has a turbidity of less than about 0.3 nephelometric turbidity units (NTU).

In another embodiment, a tube is provided. The tube includes a silicone composition, the silicone composition including a silicone matrix component, a fumed silica filler, and a vinyl-terminated silicone polymer having a viscosity of about 500 centipoise to about 5000 centipoise, wherein the tube has a turbidity of less than about 0.3 nephelometric turbidity units (NTU).

In another exemplary embodiment, a method of forming an article is provided. The method includes blending a silicone matrix component, a fumed silica filler, and a vinyl-terminated silicone polymer having a viscosity of about 500 centipoise to about 5000 centipoise to form a silicone composition. The method further includes forming the silicone composition into the article, wherein the article has a turbidity of less than about 0.3 nephelometric turbidity units (NTU).

### DETAILED DESCRIPTION

The following description is provided to assist in understanding the teachings disclosed herein. The following discussion will focus on specific implementations and embodiments of the teachings. This focus is provided to assist in describing the teachings and should not be interpreted as a limitation on the scope or applicability of the teachings. However, other teachings can certainly be used in this application.

As used herein, the terms “comprises,” “comprising,” “includes,” “including,” “has,” “having” or any other variation thereof, are intended to cover a non-exclusive inclusion. For example, a method, article, or apparatus that comprises a list of features is not necessarily limited only to those features but may include other features not expressly listed or inherent to such method, article, or apparatus. Further, unless expressly stated to the contrary, “or” refers to an inclusive-or and not to an exclusive-or. For example, a condition A or B is satisfied by any one of the following: A is true (or present) and B is false (or not present), A is false (or not present) and B is true (or present), and both A and B are true (or present).

Also, the use of “a” or “an” is employed to describe elements and components described herein. This is done merely for convenience and to give a general sense of the scope of the invention. This description should be read to include one or at least one and the singular also includes the plural, or vice versa, unless it is clear that it is meant otherwise. For example, when a single item is described herein, more than one item may be used in place of a single item. Similarly, where more than one item is described herein, a single item may be substituted for that more than one item.

Unless otherwise defined, all technical and scientific terms used herein have the same meaning as commonly understood by one of ordinary skill in the art to which this invention belongs. The materials, methods, and examples are illustrative only and not intended to be limiting. To the extent not described herein, many details regarding specific materials and processing acts are conventional and may be found in reference books and other sources within the structural arts and corresponding manufacturing arts. Unless indicated otherwise, all measurements are at about 25° C. For instance, values for viscosity are at 25° C., unless indicated otherwise.

The disclosure generally relates to a silicone article formed from a silicone composition. In an embodiment, the silicone composition includes a silicone matrix component, a fumed silica filler; and a vinyl-terminated silicone polymer. In a particular embodiment, the silicone composition may be used to form any reasonable article envisioned, such as a tube. In a particular embodiment, the silicone composition provides a silicone article with improved physical properties, such as a low level of extractables. For instance, the silicone article has a turbidity of less than about 0.3 nephelometric turbidity units (NTU), when the silicone composition is tested using solvent conditions as described by European Pharmacopoeia (EP) 3.1.9 with a 5 hour extraction in boiling water tested in a Hach 2100N turbiditimeter. Further, a method of forming a silicone article is provided.

A typical silicone composition includes a silicone matrix component. An exemplary silicone matrix component includes a polyalkylsiloxane. Any reasonable polyalkylsiloxane is envisioned. Polyalkylsiloxanes include, for example, silicone polymers formed of a precursor, such as dimethylsiloxane, diethylsiloxane, dipropylsiloxane, methylethylsiloxane, methylpropylsiloxane, or combinations thereof. In a particular embodiment, the polyalkylsiloxane includes a polydialkylsiloxane, such as polydimethylsiloxane (PDMS). In a particular embodiment, the polyalkylsiloxane is a silicone hydride-containing polyalkylsiloxane, such as a silicone hydride-containing polydimethylsiloxane. In a further embodiment, the polyalkylsiloxane is a vinyl-containing polyalkylsiloxane, such as a vinyl-containing polydimethylsiloxane. The vinyl group may be an endblock of the polyalkylsiloxane, on chain of the polyalkylsiloxane, or any combination thereof. In an embodiment, any vinyl content for the silicone matrix component is envisioned. For instance, the vinyl content for the silicone matrix component is typically about 0.006 weight % to about 0.2 weight %, based on the vinyl-containing polyalkylsiloxane, up to about 1.6 weight % based on the vinyl-containing polyalkylsiloxane, such as about 0.006 weight % to about 1.6 weight % based on the vinyl-containing polyalkylsiloxane. In yet another embodiment, the silicone matrix component is a combination of a hydride-containing polyalkylsiloxane and a vinyl-containing polyalkylsiloxane. In an example, the polyalkylsiloxane is non-polar and is free of halide functional groups, such as chlorine and fluorine, and of phenyl functional groups. Alternatively, the polyalkylsiloxane may include halide functional groups or phenyl functional groups.

The silicone matrix component further includes a catalyst. In an embodiment, any catalyst is envisioned that initiates cure of the silicone matrix component. Any reasonable catalyst that can initiate crosslinking when exposed to a cure source is envisioned. In a particular embodiment, the catalytic reaction includes aliphatically unsaturated groups reacted with Si-bonded hydrogen in order to convert an addition-crosslinkable silicone composition into an elastomeric state by formation of a network. The catalyst is activated by the cure source and initiates the crosslinking process. In an embodiment, a hydrosilylation reaction catalyst may be used. For instance, an exemplary hydrosilylation catalyst is an organometallic complex compound of a transition metal. In an embodiment, the catalyst includes platinum, rhodium, ruthenium, the like, or combinations thereof. In a particular embodiment, the catalyst includes platinum. Other exemplary catalysts may include peroxide, tin, or combinations thereof. Any catalyst or combination thereof may be envisioned as well as any amount of catalyst may be envisioned, depending upon the affect of the catalyst on the silicone matrix component as well as the processing conditions. For instance, the catalyst or combination thereof may be manipulated by varying the amount, catalyst chosen, or combination thereof to adjust the reaction rate of the silicone matrix component.

Prior to cure, the silicone matrix component has a viscosity of greater than about 50,000 centipoise, such as about 50,000 centipoise (cPs) to about 100,000,000 cPs. In an embodiment and prior to cure, the silicone matrix component has a viscosity of about 200,000 cPs to about 2,000,000 cPs, such as about 300,000 cPs to about 1,000,000 cPs. In an embodiment and prior to cure, the silicone matrix component has a viscosity of greater than about 2,000,000, such as about 2,000,000 centipoise to about 100,000,000 centipoise, such as about 2,000,000 centipoise to about 60,000,000 centipoise, such as about 8,000,000 centipoise to about 45,000,000 centipoise. The silicone matrix component may be a room temperature vulcanizable (RTV) formulation or a gel, a high consistency gum rubber (HCR), a liquid silicone rubber (LSR), or a combination thereof. In an example, the silicone matrix component is an HCR, such as SE6035, SE6075 available from Momentive, MF135 available from Bluestar silicone, and Silastic® Q7-4535, Silastic® Q7-4550 available from Dow Corning.

In a particular embodiment, the silicone matrix component is a liquid silicone rubber (LSR). In a further embodiment, the silicone matrix component is an LSR formed from a two-part reactive system. The silicone matrix component may be a conventional, commercially prepared silicone base polymer. The commercially prepared silicone base polymer typically includes the polyalkylsiloxane, the catalyst, a filler, and optional additives. Particular embodiments of conventional, commercially prepared LSR include Wacker Elastosil® LR 3003/50 by Wacker Silicone of Adrian, Mich. and Silbione® LSR 4340 by Bluestar Silicones of Ventura, Calif.

In an exemplary embodiment, a commercially prepared silicone base polymer used as the silicone matrix component is available as a one-part or two-part reactive system. With a two-part reactive system, part 1 typically includes a vinyl-containing polydialkylsiloxane, a filler, and a catalyst. Part 2 typically includes a hydride-containing polydialkylsiloxane and optionally, a vinyl-containing polydialkylsiloxane and other additives. A reaction inhibitor may be included in Part 1 or Part 2. Mixing Part 1 and Part 2 by any suitable mixing method produces the silicone matrix component. With a one-part system or two-part system, the fumed silica filler and the vinyl-terminated silicone polymer are typically added to the commercially prepared silicone matrix component prior to vulcanization to form the improved silicone composition. In an embodiment, the fumed silica filler and the vinyl-terminated silicone polymer are added to the mixed two-part system or during the process of mixing the two-part system prior to vulcanization.

Any reasonable amount of silicone matrix component is envisioned for the silicone composition. The silicone matrix component provides the base material for the silicone composition. In an embodiment, the silicone matrix component is a majority portion of the silicone composition, such as greater than about 50 weight %, based on the total weight of the silicone composition. In a particular embodiment, the silicone matrix component is present at an amount of up to about 80 weight %, such as about 50 weight % to about 80 weight %, such as about 60 weight % to about 80 weight %, based on the total weight of the silicone composition.

Further included within the silicone composition is the fumed silica filler. In a more particular embodiment, the fumed silica filler includes a surface-treated fumed silica. Any surface treatment is envisioned that provides at least one benefit of, for example, enhanced adhesion, enhanced homogeneity, reduced hydrogen bonding to the silicone composition, or any combination thereof. In an embodiment, the surface treatment of the silica includes treatment with a siloxane, such as a cyclic siloxane, a linear siloxane, or combination thereof. In a particular embodiment, any cyclic siloxane is envisioned, such as octamethylcyclotetrasiloxane (also known by those skilled in the art as “D4”). In an embodiment, any linear siloxane is envisioned, such as a polydimethyl siloxane. In an embodiment, the silica filler has a surface area that is advantageous for homogenous distribution within the silicone composition. In a particular embodiment, the fumed silica filler has a surface area of about 90 meter²/gram to about 400 meter²/gram, such as about 120 meter²/gram to about 350 meter²/gram, such as about 150 meter²/gram to about 320 meter²/gram.

Additionally, the fumed silica filler is present in any reasonable amount. For instance, the fumed silica filler is present at up to about 50% by weight, such as about 10% by weight to about 50% by weight, such as about 10% by weight to about 40% by weight, or even about 15% by weight to about 35% by weight of the total weight of the silicone composition. In an embodiment, the fumed silica filler is incorporated into the silicone composition. Any method of incorporating the fumed silica filler within the silicone composition is envisioned. In an embodiment, the fumed silica filler is dispersed in any portion of the silicone composition. In an exemplary embodiment, the fumed silica filler is typically dispersed within the silicone matrix component, and is typically mono-dispersed, being substantially agglomerate free. In another embodiment, the fumed silica filler is dispersed within the silicone matrix component as aggregates and agglomerates. An exemplary treated and fumed silica filler is available from Evonik Corporation.

In an exemplary embodiment, the silicone composition further includes a vinyl-terminated silicone polymer. “A vinyl-terminated silicone polymer” as used herein refers to a silicone polymer with vinyl functional groups at the end-block of the silicone polymer. In an embodiment, the vinyl functional group may also be present as a side group of the silicone polymer, i.e. “on-chain”. Any amount of vinyl content is present, such as a total vinyl content of up to about 1.5 weight %, such as about 0.006 weight % to about 1.5 weight %, such as about 0.006 weight % to about 0.5 weight %, such as about 0.008 weight % to about 0.25 weight %, or about 0.08 weight % to about 0.20 weight %, based on the total weight of the vinyl-terminated silicone polymer. In an embodiment, the vinyl-terminated silicone polymer is a polyalkylsiloxane, having at least one vinyl functional group at the end-block of the silicone polymer. In a particular embodiment, the vinyl-terminated silicone polymer is a polyalkylsiloxane, having at least two vinyl functional groups at the end-block of the silicone polymer. In an embodiment, the vinyl-terminated silicone polymer can include a blend of a silicone polymer using both vinyl end-blocked and vinyl on-chain polymers, with the proviso that the viscosity of the vinyl-terminated silicone polymer allows the silicone composition to be processed in conventional production equipment. In a particular embodiment, the vinyl-terminated silicone polymer has a viscosity of not greater than about 5000 centipoise, such as about 500 centipoise to about 5000 centipoise. Any reasonable amount of vinyl-terminated silicone polymer is envisioned. For instance, the vinyl-terminated silicone polymer is present at an amount of up to about 15 weight %, such as about 2.0 weight % to about 15 weight %, such as about 2.0 weight % to about 13.0 weight %, based on the total weight of the silicone composition.

The silicone composition may further include an additive. Any reasonable additive is envisioned. Exemplary additives may include, individually or in combination, a silicone gum, a hydride, a filler, an initiator, an inhibitor, a colorant, a pigment, a carrier material, or any combination thereof. In an embodiment, the inhibitor may be 1-Ethynylcyclohexanol (ETCH). In an embodiment, the additive includes a silicone gum. In a particular embodiment, the silicone gum is a vinyl-containing silicone polymer gum having a vinyl content of about 2.5 weight % to about 14 weight %, based on the total weight of the vinyl-containing silicone polymer gum. For instance, the silicone gum is a polyalkylsiloxane based silicone polymer gum having a viscosity of about 250,000 centipoise to about 2,000,000 centipoise. In a particular embodiment, the additional silicone gum may be used to modify the physical properties of the cured, final silicone article, such as increase the tear and durometer of the cured, final silicone article compared to a cured silicone article without the additional silicone gum. Any amount of the silicone gum is envisioned, such as up to about 15 weight %, such as about 0 weight % to about 15 weight %, or even about 2 weight % to about 15 weight %, based on the total weight of the silicone composition.

In an embodiment, the material content of the silicone article is essentially 100% silicone composition. In some embodiments, the silicone composition consists essentially of the respective silicone matrix component, the fumed silica filler, and the vinyl-terminated silicone polymer described above. As used herein, the phrase “consists essentially of” used in connection with the silicone composition precludes the presence of non-silicone polymers that affect the basic and novel characteristics of the silicone composition, although, commonly used processing agents and additives may be used in the silicone composition.

In an embodiment, the silicone composition is substantially free of components that would leach from the final silicone article to increase the turbidity measurement of any solvent, such as a polar solvent, for example, water, in contact with the silicone article. For instance, the silicone composition is substantially free of a filler that is treated with a hexamethyldisilazane. Further, the silicone composition is substantially free of low molecular weight silicone, such as silanols having a molecular weight of less than about 1,200 g/mol. “Substantially free” as used herein refers to a silicone composition that has less than about 1.0% by weight of the total weight of the silicone composition.

In the embodiment, a method of making an article is included. The method includes any reasonable method for extruding, molding, laminating, or coating the article. The method includes blending the silicone matrix component, the fumed silica filler, and the vinyl-terminated silicone polymer to form the silicone composition. Any method of blending the components of the silicone composition is envisioned. In an example, the mixing device is a mixer in an injection molder. In another example, the mixing device is a mixer, such as a dough mixer, Ross mixer, two-roll mill, or internal mixer, banbary mixer, or Brabender mixer. The mixer can melt and/or mix components of the silicone composition. In an embodiment, the components of the silicone composition can be provided to the mixer in the form of a liquid, a solid, such as pellets, strips, powders, and the like, or any combination thereof. Once the components of the silicone composition are mixed, the silicone composition may be delivered to any reasonable apparatus for formation into the article, such as an injection molding device. A typical molding device includes a mold having a cavity configured in any shape desired for the final injection molded article. The silicone composition can then be cured and post-processed by any reasonable method and apparatus.

In an embodiment, the mixing device is a mixer for an extrusion apparatus. Typically, the mixing device for an extrusion apparatus includes a pumping system. The pumping system can include a number of devices that can be utilized to form the silicone article. For example, the pumping system can include a pumping device such as a gear pump, a static mixer, an extrusion device, a cure device, a post-processing device, or any combination thereof. The pumping system may include any reasonable means to deliver the components of the silicone composition such as pneumatically, hydraulically, gravitationally, mechanically, and the like, or combinations thereof. In an embodiment, the extrusion apparatus can include an extruder, such as a single screw extruder or a twin screw extruder. The extruder can melt and/or mix components of the silicone composition. In an embodiment, the components of the silicone composition can be provided to the extruder in the form of a liquid, a solid, such as pellets, strips, powders, and the like, or any combination thereof. Once mixed and extruded, the silicone composition can also be cured and post-processed by any reasonable method and apparatus.

In an illustrative embodiment, the extrusion apparatus is organized such that one or more components of the system are arranged in a vertical configuration. For example, the extruder, the die, and the components of an energy source to cure the silicone composition are arranged to vertically extrude the silicone article. In a particular embodiment, the silicone article can be formed by extruding the silicone composition in an upward direction or a downward direction. In a more particular embodiment, the silicone article is formed by extruding the silicone composition in an upward direction. In an example, the vertical upward extrusion may provide increased dimensional stability to the final silicone article. In an alternative embodiment, the extrusion apparatus can be arranged in a horizontal configuration.

In an embodiment, the silicone composition is subjected to any source of energy to cure the silicone composition to form the silicone article. Cure may occur at any step of the process once the components of the silicone composition are mixed. The source of energy can include any reasonable energy source such as heat, radiation, or combination thereof. The energy source includes vulcanizing the silicone composition at any reasonable conditions to substantially cure the silicone composition. “Substantially cure” as used herein refers to >90% of final crosslinking density, as determined for instance by rheometer data (90% cure means the material reaches 90% of the maximum torque as measured by ASTM D5289). For instance, the level of cure is to provide a silicone article having a desirable shore A durometer. Any shore A durometer is envisioned, such as less than about 80, such as about 10 to about 80, such as about 20 to about 80, such as about 20 to 70, or even about 40 to about 70. In an embodiment, cure conditions may vary depending on the source of cure as well as the components of the silicone composition.

When the source of energy is heat, cure may be at any reasonable temperature for any reasonable time. Thermal cure typically occurs at a temperature of about 125° C. to about 200° C. In an embodiment, the thermal treatment is at a temperature of about 150° C. to about 200° C. Typically, the thermal treatment occurs for any time period, such as a time period of about 2 seconds to about 15 minutes, such as about 10 seconds to about 10 minutes, or even about 10 seconds to about 5 minutes. For instance, cure, i.e. vulcanization, of the silicone composition includes heat of about 125° C. to about 200° C. for a time of about 3 seconds to about 15 minutes.

When the source of energy includes radiation energy, any reasonable radiation energy source is envisioned, such as actinic radiation. In a particular embodiment, the radiation source is ultraviolet light. In an example, the radiation source is sufficient to substantially cure the silicone article. Any reasonable wavelength of ultraviolet light is envisioned. In a specific embodiment, the ultraviolet light is at a wavelength of about 10 nanometers to about 500 nanometers, such as a wavelength of about 200 nanometers to about 400 nanometers. Cure conditions may be dependent upon the components of the silicone composition. Further, any combination of wavelengths may be envisioned, such as one or more ultraviolet application of the same or different wavelength.

The cured silicone article can undergo post processing. Any post processing is envisioned. In an embodiment, the post processing can include a heating tower. For instance, the silicone article can be subjected to a heat treatment, such as a post-curing heat treatment. Any post-curing heating treatment may be envisioned. A typical post-curing heat treatment includes a temperature of greater than about 100° C., such as about 100° C. to about 250° C., such as about 100° C. to about 220° C., such as about 100° C. to about 200° C., for any reasonable time period, such as about 1 hour to 10 hours. In an alternative example, the silicone article is not subjected to a post-cure heat treatment. In an example, the silicone article can include a silicone tube structure that is post processed by cutting the silicone tube into a number of silicone tubes having a specified length. Although a typical extrusion apparatus, injection molding apparatus, cure, and process is described, any variations may be envisioned that blends the components of the silicone composition and cures the silicone composition via an energy source.

Any reasonable silicone article may be formed with the silicone composition. For instance, any silicone article may be envisioned. In a particular embodiment, the silicone article is a film, a block, a circular tube, a rectangular tube, a shaped profile of either open or closed geometry, and the like. An exemplary profile includes, but is not limited to, gaskets, seals, medical components, laboratory septa and multilumens. In an embodiment, the silicone article is a tube. A tube typically includes a proximal end, a distal, and a lumen there through. The proximal end to the distal end defines a length of the tube. The tube further includes an inner diameter that defines an inner surface of the tube and an outer diameter that defines an outside surface of the tube. The article may include any number of layers. In an embodiment, a multilayer article is produced such as a film, tubing, and the like. In an embodiment, the silicone composition may be combined with additional materials such as reinforcements, tie layers, and the like. Any material used for a multilayer article is envisioned depending on the final article desired. The article may also include a foamed structure.

The silicone compositions and the method of making a silicone article may advantageously produce low durometer silicone elastomers having desirable physical and mechanical properties. Typically, the silicone article is elastomeric. For example, the durometer (Shore A) of the final silicone article may be less than about 80, such as about 10 to about 80, such as about 20 to about 80, such as about 20 to 70, or even about 40 to about 60. Further advantageous physical properties include for example, improved elongation-at-break, tensile strength, or tear strength. Elongation-at-break and tensile strength are determined using an Instron instrument in accordance with ASTM D-412 testing methods. For example, the silicone article may exhibit an elongation-at-break of at least about 250%, such as at least about 350%. In an embodiment, the tensile strength of the silicone article is greater than about 1000 psi. Further, the silicone article may have a tear strength greater than about 150 ppi for a silicone article having a shore A durometer of about 25 to about 75.

The silicone article has an advantageous level of extractables. For instance, the turbidity measurement of the silicone article is less than about 0.30 nephelometric turbidity units (NTU), such as less than about 0.25 NTU, such as less than about 0.20 NTU, without any post-cure of the silicone article. When the silicone article has been subjected to a post-cure, the turbidity measurement is less than about 0.10 nephelometric turbidity units (NTU), such as less than about 0.08 NTU, such as less than about 0.05 NTU. The turbidity measurements are taken in accordance with EP 3.1.9.

Advantageously, the silicone composition in conjunction with the processing of the silicone composition may provide silicone articles that are not achieved by conventional silicone compositions and manufacturing processes. In a particular embodiment, the use of a radiation cure and the operating parameters for the components of the pumping system are conducive to forming dimensionally accurate tubing that conventional extrusion/heat cure systems are not able to re-produce. For instance, the use of a radiation source typically cures the silicone article more rapidly compared to conventional heat cure systems. “Conventional heat cure” as used herein refers to curing via heat at a temperature greater than about 120° C. Although not being bound by theory, it is believed that the radiation cure provides instant penetration of the radiation into the silicone composition and curing of the bulk of the silicone composition concurrently. Additionally, arranging an extrusion apparatus such that the tubing is extruded in a vertical direction may contribute to reducing variation in the dimensions of the tubing.

Types of applications for the silicone article include as examples, but not limited to, any application where low extractables for the silicone article are desired. For instance, applications may be for the medical industry, pharmaceutical industry, biopharmaceutical industry, a health care industry, food and beverage industry, electronic industry, and the like. Exemplary articles include molded devices, extruded devices, surgical drains, piston valves, intravenous applications, catheters, flexible tubing, sealants, and the like.

Many different aspects and embodiments are possible. Some of those aspects and embodiments are described herein. After reading this specification, skilled artisans will appreciate that those aspects and embodiments are only illustrative and do not limit the scope of the present invention. Embodiments may be in accordance with any one or more of the items as listed below.

ITEMS

Item 1. A silicone article including a silicone composition, the silicone composition including a silicone matrix component; a fumed silica filler; and a vinyl-terminated silicone polymer having a viscosity of about 500 centipoise to about 5000 centipoise, wherein the silicone article has a turbidity of less than about 0.3 nephelometric turbidity units (NTU).

Item 2. A tube including a silicone composition, the silicone composition including: a silicone matrix component; a fumed silica filler; and a vinyl-terminated silicone polymer having a viscosity of about 500 centipoise to about 5000 centipoise, wherein the tube has a turbidity of less than about 0.3 nephelometric turbidity units (NTU).

Item 3. A method of forming an article including: blending a silicone matrix component, a fumed silica filler, and a vinyl-terminated silicone polymer having a viscosity of about 500 centipoise to about 5000 centipoise to form a silicone composition; and forming the silicone composition into the article wherein the article has a turbidity of less than about 0.3 nephelometric turbidity units (NTU).

Item 4. The silicone article, the tubing, and the method of any of the preceding Items, wherein the silicone matrix component has a viscosity of greater than about 50,000 centipoise, such as about 50,000 centipoise to about 100,000,000 centipoise.

Item 5. The silicone article, the tubing, and the method of any of the preceding Items, wherein the silicone matrix component is a vinyl-containing polyalkylsiloxane.

Item 6. The silicone article, the tubing, and the method of any of the preceding Items, wherein the silicone matrix component is platinum-catalyzed.

Item 7. The silicone article, the tubing, and the method of any of the preceding Items, wherein the fumed silica filler is treated with a cyclic siloxane, a polydimethyl siloxane, or a combination thereof.

Item 8. The silicone article, the tubing, and the method of any of the preceding Items, wherein the fumed silica filler has a surface area of about 120 meter²/gram to about 350 meter²/gram.

Item 9. The silicone article, the tubing, and the method of any of the preceding Items, wherein the vinyl-terminated silicone polymer has a total vinyl content of up to about 1.5 weight %, such as about 0.008 weight % to about 0.2 weight %.

Item 10. The silicone article, the tubing, and the method of any of the preceding Items, wherein the silicone matrix component is present at an amount greater than about 50 weight %, such as about 60 weight % to about 80 weight %, based on the total weight of the silicone composition.

Item 11. The silicone article, the tubing, and the method of any of the preceding Items, wherein the fumed silica filler is present at an amount of about 15 weight % to about 35 weight %, based on the total weight of the silicone composition.

Item 12. The silicone article, the tubing, and the method of any of the preceding Items, wherein the vinyl-terminated silicone polymer is present at an amount of up to about 15 weight %, such as about 2.0 weight % to about 13.0 weight %, based on the total weight of the silicone composition.

Item 13. The silicone article, the tubing, and the method of any of the preceding Items, wherein the silicone composition further includes a hydride fluid, an inhibitor, a silicone gum, or a combination thereof.

Item 14. The silicone article, the tubing, and the method of any of the preceding Items, wherein the silicone composition has a shore A durometer of about 20 to about 80.

Item 15. The silicone article, the tubing, and the method of any of the preceding Items, wherein the silicone composition can be used for a medical application, a biopharmaceutical application, a pharmaceutical application, a health care application, or any combination thereof.

Item 16. The silicone article or method of Item 1 and 3, wherein the article is a tube.

Item 17. The method of Item 3, further including curing the silicone composition by thermal cure, radiation cure, or combination thereof.

Item 18. The method of Item 17, wherein curing the silicone composition by thermal cure is at a temperature of about 125° C. to about 200° C. for a time of about 3 seconds to about 15 minutes.

Item 19. The method of Item 17, wherein curing the silicone composition by radiation cure is an ultraviolet cure at a wavelength of about 10 nanometers to about 500 nanometers.

Item 20. The method of Item 17, further including post curing the silicone composition at a temperature of about 100° C. to about 250° C. for a time of about 1 hour to 10 hours.

Item 21. The method of Item 20, wherein the post-cured silicone composition has a turbidity of less than about 0.1 NTU.

Item 22. The method of Item 3, wherein forming the blend into the article includes extruding, molding, laminating, or coating.

The following examples are provided to better disclose and teach processes and compositions of the present invention. They are for illustrative purposes only, and it must be acknowledged that minor variations and changes can be made without materially affecting the spirit and scope of the invention as recited in the claims that follow.

### EXAMPLES

### Example 1

Several formulations are mixed and formed into a silicone tube using the below components (amounts in weight %, based on the total weight of the silicone composition) seen in Tables 1 and 2. The silicone matrix component includes a vinyl end-blocked polyalkylsiloxane (i.e. indicated as the vinyl end-blocked gum having a vinyl content of 0.008wt %, based on the total weight of the vinyl-end blocked gum), a vinyl on-chain polyalkylsiloxane (i.e. indicated as the vinyl on-chain gum having a vinyl content of 0.2wt %, based on the totally weight of the vinyl on-chain gum), or combination thereof. The silicone matrix component has a viscosity of greater than 1,000,000 cps. The treated filler is silica filler treated with a cyclic siloxane, a polydimethyl siloxane, or a combination thereof. The vinyl-terminated silicone polymer is indicated as the vinyl stopped fluid, with the viscosity as indicated. Optional additives include a hydride fluid and a silicone gum that is a vinyl-containing silicone polymer gum (i.e. indicated as the high vinyl gum). Cure conditions are indicated in Tables 1 and 2 of 10 minutes at 350° F. (177° C.). Elongation-at-break and tensile strength are determined using an Instron instrument in accordance with ASTM D-412 testing methods. Shore A durometer (hardness), and tear strength are also measured by ASTM 2240 and ASTM 412, respectively. Further, the turbidity is measured in accordance with EP 3.1.9.

TABLE 1

| | Formulation 1 | Formulation 2 |
|---|---|---|
| Cure conditions | 10 minutes at 350 F. | 10 minutes at 350 F. |
| 0.008% vinyl endblocked gum | 76.5 | 0 |
| 0.2% vinyl on chain gum | 0 | 64 |
| Treated filler | 20 | 32 |
| Catalyst | 10 ppm platinum | 10 ppm platinum |
| 1,000 cps vinyl stopped fluid | 3 | 3 |
| Hydride fluid | 0.5 | 1 |
| Tensile Psi | 1690 | 1483 |
| Elongation % | 831 | 425 |
| Hardness | 36 | 59 |
| Die B tear | 150 | 140 |
| NTU | 0.21 | 0.23 |
| NTU post cured 2 hrs @400 F. | 0.08 | 0.05 |

TABLE 2

| | Formulation 3 | Formulation 4 |
|---|---|---|
| Cure conditions | 10 minutes at 350 F. | 10 minutes at 350 F. |
| | | 2 hrs at 400 F. post cure |
| 0.008% vinyl endblocked gum | 16 | 16 |
| 0.2% vinyl on chain gum | 48.75 | 48.75 |
| Treated filler | 29 | 29 |
| Catalyst | 10 ppm platinum | 10 ppm platinum |
| 5,000 cps vinyl stopped fluid | 3 | 3 |
| Hydride fluid | 1.25 | 1.25 |
| High vinyl gum | 2 | 2 |
| Tensile psi | 1342 | 1365 |
| Elongation % | 450 | 428 |
| Hardness | 62 | 65 |
| Die B tear | 250 | 201 |
| NTU | 0.18 | 0.09 |

Clearly, Formulations 1, 2, and 3 provide samples with turbidity values less than 0.3 NTU, with and without post cure. Formulation 4 is the same formulation as Formulation 3, with the added post-cure of 400° F. (204° C.) for 2 hours. Post curing of the formulations indicates that post curing can improve the NTU value, with a turbidity values decreasing after post cure to less than 0.1 NTU.

### Example 2

Commercial samples are tested for comparison to the silicone compositions of Example 1. Bases are cured with 1 part of hydride and 10 ppm platinum and are industrial and medical grade silicone compositions used for tubing and molded articles. Grade and Supplier are listed below with turbidity results. Samples indicated as “post cured” are exposed to heat at 400° F. for 2 hours.

TABLE 3

| | NTU units | |
|---|---|---|
| DC Q7-4750 A&B | 0.56 | Dow Corning |
| DC Q7-4750 A&B post cured | 0.38 | Dow Corning |
| SE 94500 BASE | 0.58 | Momentive |
| ShinEtu 74000 U | 0.72 | ShinEtu |
| ShinEtu 45010 A&B | 0.78 | ShinEtu |
| Shin Etu 43010 A&B | 0.59 | ShinEtu |
| Wacker 401-40 BASE | 0.77 | Wacker |
| Wacker 401-60 BASE | 0.68 | Wacker |
| Wacker 4305-70 A&B | 0.65 | Wacker |
| DC Q7-4730 A&B | 0.59 | Dow Corning |
| DC Q7-4730 A&B post cured | 0.46 | Dow Corning |

Clearly, Formulations 1, 2, 3, and 4 of Example 1 have lower turbidity values compared to commercially available formulations. In particular, Formulations 1, 2, 3, and 4 of Example 1 have turbidity values less than about 0.3 NTU, whereas the commercially available formulations have turbidity values greater than 0.38 NTU. The samples obtained from Dow Corning have the lowest turbidity values at 0.56 NTU and 0.59 NTU, respectively. However, even post cure of the samples from Dow Corning does not provide turbidity values as the current formulations provide.

Note that not all of the activities described above in the general description or the examples are required, that a portion of a specific activity may not be required, and that one or more further activities may be performed in addition to those described. Still further, the order in which activities are listed is not necessarily the order in which they are performed.

Benefits, other advantages, and solutions to problems have been described above with regard to specific embodiments. However, the benefits, advantages, solutions to problems, and any feature(s) that may cause any benefit, advantage, or solution to occur or become more pronounced are not to be construed as a critical, required, or essential feature of any or all the claims.

The specification and illustrations of the embodiments described herein are intended to provide a general understanding of the structure of the various embodiments. The specification and illustrations are not intended to serve as an exhaustive and comprehensive description of all of the elements and features of apparatus and systems that use the structures or methods described herein. Separate embodiments may also be provided in combination in a single embodiment, and conversely, various features that are, for brevity, described in the context of a single embodiment, may also be provided separately or in any subcombination. Further, reference to values stated in ranges includes each and every value within that range. Many other embodiments may be apparent to skilled artisans only after reading this specification. Other embodiments may be used and derived from the disclosure, such that a structural substitution, logical substitution, or another change may be made without departing from the scope of the disclosure. Accordingly, the disclosure is to be regarded as illustrative rather than restrictive.

What is claimed is:

1. A silicone article comprising a silicone composition, the silicone composition comprising:
a silicone matrix component;
a fumed silica filler; and
a vinyl-terminated silicone polymer having a viscosity of about 500 centipoise to about 5000 centipoise, wherein the silicone article has a turbidity of less than about 0.3 nephelometric turbidity units (NTU).

2. A tube comprising a silicone composition, the silicone composition comprising:
a silicone matrix component;
a fumed silica filler; and
a vinyl-terminated silicone polymer having a viscosity of about 500 centipoise to about 5000 centipoise, wherein the tube has a turbidity of less than about 0.3 nephelometric turbidity units (NTU).

3. A method of forming an article comprising:
blending a silicone matrix component, a fumed silica filler, and a vinyl-terminated silicone polymer having a viscosity of about 500 centipoise to about 5000 centipoise to form a silicone composition; and
forming the silicone composition into the article wherein the article has a turbidity of less than about 0.3 nephelometric turbidity units (NTU).

4. The silicone article of claim 1, wherein the silicone matrix component has a viscosity of greater than about 50,000 centipoise, such as about 50,000 centipoise to about 100,000,000 centipoise.

5. The silicone article of claim 1, wherein the silicone matrix component is a vinyl-containing polyalkylsiloxane.

6. The silicone article of claim 1, wherein the silicone matrix component is platinum-catalyzed.

7. The silicone article of claim 1, wherein the fumed silica filler is treated with a cyclic siloxane, a polydimethyl siloxane, or a combination thereof.

8. The silicone article of claim 1, wherein the fumed silica filler has a surface area of about 120 meter²/gram to about 350 meter²/gram.

9. The silicone of claim 1, wherein the vinyl-terminated silicone polymer has a total vinyl content of up to about 1.5 weight %, such as about 0.008 weight % to about 0.2 weight %.

10. The silicone article of claim 1, wherein the silicone matrix component is present at an amount greater than about 50 weight %, such as about 60 weight % to about 80 weight %, based on the total weight of the silicone composition.

11. The silicone article of claim 1, wherein the fumed silica filler is present at an amount of about 15 weight % to about 35 weight %, based on the total weight of the silicone composition.

12. The silicone article of claim 1, wherein the vinyl-terminated silicone polymer is present at an amount of up to about 15 weight %, such as about 2.0 weight % to about 13.0 weight %, based on the total weight of the silicone composition.

13. The silicone article of claim 1, wherein the silicone composition further comprises a hydride fluid, an inhibitor, a silicone gum, or a combination thereof.

14. The silicone article of claim 1, wherein the silicone composition has a shore A durometer of about 20 to about 80.

15. The silicone article of claim 1, wherein the silicone composition can be used for a medical application, a biopharmaceutical application, a pharmaceutical application, a health care application, or any combination thereof.

16. The silicone article of claim 1, wherein the article is a tube.

17. The method of claim 3, further comprising curing the silicone composition by thermal cure, radiation cure, or combination thereof.

18. The method of claim 17, wherein curing the silicone composition by radiation cure is an ultraviolet cure at a wavelength of about 10 nanometers to about 500 nanometers.

19. The method of claim 17, further comprising post curing the silicone composition at a temperature of about 100° C. to about 250° C. for a time of about 1 hour to 10 hours.

20. The method of claim 19, wherein the post-cured silicone composition has a turbidity of less than about 0.1 NTU.
